(19) United States
(12) Patent Application Publication
(10) Pub. No.: US 2017/0312504 A1
(43) Pub. Date: Nov. 2, 2017

(54) IMPLANT FOR USE IN THE CARDIOVASCULAR SYSTEM

(71) Applicant: Ameber Medical LTD, Jerusalem (IL)

(72) Inventors: Michael Fishman, Ashkelon (IL); Alexander Tsun, Ashdod (IL)

(21) Appl. No.: 15/650,925

(22) Filed: Jul. 16, 2017

Related U.S. Application Data
(63) Continuation-in-part of application No. 14/375,435, filed on Jul. 30, 2014.
(60) Provisional application No. 61/592,013, filed on Jan. 30, 2012.
(60) Provisional application No. 61/674,861, filed on Jul. 24, 2012.

(51) Int. Cl. A61N 1/10; A61F 2/00; A61L 31/08; A61F 2/82; C23C 14/34; C23C 14/08

(52) U.S. Cl. CPC A61N 1/10; C23C 14/3407; C23C 14/083; A61L 31/088; A61F 2/82; A61F 2/0077; A61F 2240/001; A61L 2430/20

(57) ABSTRACT

An implant for the cardiovascular system is provided, the implant is insertable into an organ and includes a body structure configured to be disposed inside an organ; and an electret coating disposed on the body structure; wherein the electret coating includes a negative charge such that a negative electrostatic field is formed in proximity of the body structure, the charge is such that the negative electrostatic field corresponds to a positive electrostatic field formed by a damaged tissue of the organ.

This application is a continuation-in-part application of U.S. patent application Ser. No. 14/375,435 filed on Jul. 30, 1014.

### FIELD OF INVENTION

The presently disclosed subject matter relates to an implant for use in the cardiovascular system, in general, and in particular to a cardiovascular implant for insertion in a blood carrying organ.

### BACKGROUND

A stent is an artificial element inserted into a natural passage/conduit in the body to prevent, or counteract, a disease-induced, localized flow constriction. The stents are inserted into narrowed coronary arteries to help keep them open after balloon angioplasty. The stent then allows the normal flow of blood and oxygen to the heart. Stents can be placed in narrowed carotid arteries (the vessels in the front of the neck that supply blood to the brain) appear useful in treating patients at elevated risk for stroke.

WO/1996/041589 discloses a device for implantation in a vessel or hollow organ lumen in a human or animal body, such as a self-expanding stent, a vena cava filter, an embolizing means or a supporting means, comprises a wire frame with a plurality of interconnected cells made of at least two separate wire sections which are intercrossing at cell junctions and form closed cells. At the cell junctions the wires are knot to form a geometrical locking of the cells so that the wire-shaped cell sides in respective cells are locked at the cell junctions when the wire frame is subjected to pressure acting radially inwards.

RU 2446775 discloses a stent includes a tubular element which has longitudinal axis. Tubular element contains first radial element and second radial element. First radial element contains elongated bridge with deflectable teeth. Bridge is characterized by upper and lower parts. Upper part narrows from first thickness to second thickness on first direction on circumference, approaching far edge of upper part. Lower part narrows from first thickness to second thickness in direction on circumference opposite to first direction, approaching far edge of lower part. Second radial element is located on circumference near first radial element and contains gearing means, which have configuration for sliding gearing of elongated bridge of first radial element and deflection of deflectable teeth, when teeth contact with gearing means in such a way that tubular element reaches extension in direction on circumference with reduced kickback. Gearing means narrow to second thickness in direction on circumference, approaching far edge of gearing means. In second version of stent implementation gearing means of second radial element include slot intended for sliding gearing of first radial element bridge and deflection of teeth, when elongated bridge of first radial element passes through slot, in such a way that tubular element obtains extension in direction on circumference with reduced kickback. Second radial element does not overlap itself when stent is in extended state. Gearing means narrow on circumference to second thickness, approaching far edge of gearing means in such a way, that first and second radial elements preserve mainly constant common thickness, when first radial element is geared by gearing means of second radial element and stent is in extended state.

U.S. Pat. No. 6,231,600 discloses a device such as a stent provided with a hybrid coating including a time released, restenosis inhibiting coating and a non-thrombogenic coating to prevent clotting on the device. One first coat or layer includes a polymer, a crosslinking agent, and pacitaxel, analogues, or derivatives thereof. The first coat preferably includes a polymer having Taxol admixed therein so as to be releasable over time. The first coat preferably includes a polyfunctional aziridine as the crosslinking agent. The second coat preferably includes heparin to inhibit clot formation on the device. The crosslinking agent can covalently bond to both the first coat polymer and the second coat heparin. A stent can be provided with a first coat including an aqueous dispersion or emulsion of a polymer and an excess of crosslinking agent. The first coating can be dried, leaving a water insoluble polymer coating. A second aqueous coating including a solution or dispersion of heparin can be applied over the first coating, the heparin becoming covalently bound to the crosslinking agent on the first coating surface. The resulting stent can inhibit restenosis while preventing blood clot formation on the stent.

RU 2380059 discloses a stent coating containing a polymer material with an active antiproliferative substance. The polymer material presents a copolymer of butyric and valeric acids, while the active antiproliferative substance is rubomycinum. The amount of the copolymer of butyric and valeric acids per one stent is 2-15 mg/stent, while rubomycinum composes a polymer layer in amount 0.002-0.025 mg/stent. US Patent Application 2008/0208315 disclosed a stent for coronary vessels, having a surface of multilayer immobilized structures, includes a stent body and a number of polyelectrolyte complex (PEC) layers stacking and being immobilized on the surface of the stent body, in which the PEC layer is formed of a polymer layer and an anticoagulant layer. The coronary stent is capable of effectively improving the hemocompatibility and longevity over a conventional stent using surface encapsulation of an anticoagulant layer for hemocompatibility improvement. Furthermore, the coronary stent can be use as a drug-eluting coronary stent, thus allowing for the time-releasing of drugs, and further preventing the thickening of vascular smooth muscle cells for causing vascular thrombosis.

It is known in the prior art that blood cells being components of thrombosis such as thrombocytes and leucocytes are negatively charged. US Patent Application 2006/0106451 discloses an electronic anti-coagulation stent structure. The stent structure comprises a pair of coaxial metal stents having a layer of dielectric material between the stents. A battery is operatively connected preferably near or adjacent to the upstream end upon deployment of the stent. The positive battery terminal establishes an electrical connection to the outer metal stent and the negative terminal establishes an electrical connection to the inner metal stent and this exhibits a capacitor-like properties. The inner metal stent, being negatively charged, promotes a platelet repellent, anti-thrombotic effect.

US Patent Application 2011/0196478 discloses devices and methods for lumen treatment. There is provided an endoprosthesis that includes an internal layer designed to provide a negative electric field directed endoluminally; an external layer designed to provide a positive electric field directed exoluminally; and one or more intermediate layers disposed between the internal layer and the external layer, wherein the negative electric field is due to a negative point charge between about −25 mV and about −250 mV, and wherein the positive electric field is due to a positive point charge between about +1 mV and about +30 mV.

As known in the art, damaged tissue inside the cardiovascular system is prone to stimulate the formation of restenosis and thrombosis. Restenosis is the recurrence of stenosis after a procedure it is due to Neointimal Hyperplasia which is the proliferation and migration of vascular smooth muscle cells primarily in the tunica intima, resulting in the thickening of arterial walls and decreased arterial lumen space. Neointimal hyperplasia is the major cause of restenosis after percutaneous coronary interventions, such as stenting or angioplasty.

Neointimal hyperplasia first develops with damage to the arterial wall, followed by platelet aggregation at site of injury, recruitment of inflammatory cells, proliferation and migration of vascular smooth muscle cells, and collagen deposition.

In addition, as known, thrombosis is the formation of a blood clot inside a blood vessel, obstructing the flow of blood through the circulatory system.

A thrombus, colloquially called a blood clot, is the final product of the blood coagulation step in hemostasis. There are two components to a thrombus: first is the aggregated platelets and red blood cells that form a plug, and the second is a mesh of cross-linked fibrin protein.

The Endothelium helps preventing Blood clotting (thrombosis & fibrinolysis). The endothelium normally provides a non-thrombogenic surface because it contains, for example, heparan sulfate which acts as a cofactor for activating antithrombin, a protease that inactivates several factors in the coagulation cascade.

### SUMMARY OF INVENTION

There is provided in accordance with an aspect of the presently disclosed subject matter an implant for the cardiovascular system, insertable into an organ, the implant
including a body structure configured to be disposed inside an organ; and an electret coating disposed on the body structure;
wherein the electret coating includes a negative charge such that a negative electrostatic field is formed in proximity of the body structure, the charge is such that the negative electrostatic field corresponds to a positive electrostatic field formed by a damaged tissue of the organ.

The negative electrostatic field can be configure to restore body conditions of a healthy tissue corresponding to the damaged tissue.

The body conditions includes an electrostatic field formed by a healthy tissue of an organ in which the implant is disposed.

The negative electrostatic field can be configured to compensate for the positive electrostatic charge caused by the damaged tissue and which causes a change in the overall electrostatic field in the damaged site inside the organ.

The negative charge can be configured to form a sum electrostatic field inside a segment of the organ in which the implant is mounted such that difference between the sum electrostatic field and an electrostatic field in healthy areas in close proximity to the damaged tissue precludes undesirable electric forces acting on platelets and red blood cells.

The negative charge can be configured in accordance with the size of the damaged area. The negative charge can be configured in accordance with the risk level for damaged tissue determined in accordance with the physical conditions of the patient in which the implant is mounted. The negative charge can be configured to restore an electrostatic field in the range of 31 50 mv to −400 mv. The negative charge can be selected depending on the type of organ and a degree of the damaged tissue. The negative charge can be in a range of

$$- 4 \cdot {10}^{- 8} \frac{c}{{m}^{2}} to - 4 \cdot {10}^{- 7} \frac{c}{{m}^{2}}$$

and can be selected such that the hemostasis in the organ is maintained and blood flow therein is not interrupted.

The body structure can include a plurality of wires arranged to form a cylindrical body wherein each of the wires is coated with a coating having a negative electrostatic charge forming thereby a negative electrostatic field in the inner volume of the cylindrical body, wherein the wires are arranged such that an entire area between each one of the wires has a negative electric filed.

The distance between two neighboring wires of the plurality of wires can be less than 15 times the diameter of the wire and the negative electric charge carried by each of the wire is sufficient to form a negative electric field which covers the entire area between the wires.

The electret coating can be formed by means of coating the stent with dielectric material.

A decay rate of the negative charge in the coating can correspond to the healing process of the damaged tissue such that the rate at which the coating losses the negative electric charge corresponds to the rate at which the damaged tissue losses the positive electric charge.

There is provided in accordance with another aspect of the presently disclosed subject matter a method for forming an implant for the cardiovascular system, insertable into an organ. The method includes providing a body structure configured to be disposed inside an organ; coating the body structure with an electret coating; wherein the electret coating includes a negative charge such that a negative electrostatic field is formed in proximity of the body structure, the charge is such that the negative electrostatic field corresponds to a positive electrostatic field formed by a damaged tissue of the organ.

The step of coating can be carried out with vacuum-plasma sputtering.

The vacuum-plasma sputtering can include placing the body structure inside a sputtering system which ejects particles of tantalum pentoxide onto the implant together with negatively charged particles.

The vacuum-plasma sputtering can further include forming a layer of tantalum pentoxide having defects in a crystalline structure of the layer and targeting the negatively charged particles into the defects.

The vacuum-plasma sputtering can further include forming at least a first layer and a second layer of tantalum pentoxide, such that negatively charged particles disposed in the defects of the first layer, are covered by the second layer holding the negative charge inside the coating.

The level of stability of the negative charge inside the coating can be determined in accordance with a required lifetime of the negative electrostatic filed such that the electrostatic filed compensates for the action of the positive electric charge of the damaged tissue.

The vacuum-plasma sputtering can be carried out such that temperature of the implant is maintained below a predefined threshold.

The threshold can be defined in accordance with the temperature required to maintain the negatively charged particles negatively charged particles inside the defects.

The body structure can be expandable and wherein the method further comprising expanding the body structure prior to the coating step facilitating thereby coating of all areas of the body structure with the plasma coating.

The expansion can be at a rate determined in accordance with an expansion expected to occur during installation of the implant inside the organ, such that plastic deformation of the body structure does not compromise performance of the implant.

The method can further include placing the body structure on a heat sink prior to the step of coating, wherein the heat sink can be configured to evacuate excess heat from the body structure such that the negative charge is maintained in the coating.

The implant can be a stent and the heat sink includes one or more rotating rods configured for mounting thereon the stent.

The diameter of the rod can be smaller than the inner dimeter of the stent such that the inner circumference of the wall of the stent does not fully engage the surface of the rod allowing thereby sputtered particles to reach inner surfaces of the stent.

The rotating rods are disposed at an angle with respect to a horizontal plane of the heat sink such that the stent gravitate downwardly.

The rotating rods can include a plurality of ribs extending at an angle with respect to a longitudinal axis of the rods.

The rotating rods can be anchored perpendicular to a disc. The disc can be coupled to a base including a cooling device for evacuating heat from the heat sink.

The step of coating can be carried out with vacuum-plasma sputtering in which a stream of particles is sprayed over the implant and wherein the heat sink is disposed at an angle with respect to the stream such that the particles evenly reach a longitudinal axis of the implant.

There is provided in accordance with another aspect of the presently disclosed subject matter an article insertable into a patient's body.

The article can be coated with a coating having an electret structure. A further object of the invention is to disclose the coating having an integral surface blanketing an entire surface of the wire member. A further object of the invention is to disclose the coating which is tantalum pentoxide. A further object of the invention is to disclose the tantalum pentoxide coating which is vacuum-plasma sputtering.

A further object of the invention is to disclose the vascular stent constructed of tubing cut into a mesh shape. A further object of the invention is to disclose the vascular stent constructed of at least one wire into a mesh shape.

Another object of the invention is to disclose a vascular stent carrying a negative charge. The aforesaid stent comprises at least one metal wire member configured into a cylindrical celled frame.

A further object of the invention is to disclose the stent in which a distance between each two neighboring wires of the stent is less 15 wire diameters such that the stent provides substantially uniform electrostatic field preventing blood cells from aggregation on the stent wires.

A further object of the invention is to disclose a method of manufacturing a vascular stent carrying a negative charge and insertable into a patient's body. The aforesaid method comprises the steps of: (a) providing an article work piece; and (b) coating the wire members with a coating carrying a negative charge; and

It is another core purpose of the invention to provide the step of coating article work piece further comprises creating an electret structure of the work piece.

A further object of the invention is to disclose the step of coating the wire members further comprising creating an integral surface blanketing an entire surface of the wire member.

A further object of the invention is to disclose the step of coating the wire members further comprising sputtering tantalum pentoxide.

A further object of the invention is to disclose the step of sputtering tantalum pentoxide which is performed by means of vacuum-plasma sputtering.

As used therein, the term “cardiovascular implant” is to be interpreted as including: vascular stent, an artificial cardiac valve stent, vascular graft, Left Atrial Appendage Occlusion stent, MitraClip, ASD stents and other congenital heart defect clips and stents and the like.

### BRIEF DESCRIPTION OF THE DRAWINGS

In order to understand the disclosure and to see how it may be carried out in practice, embodiments will now be described, by way of non-limiting examples only, with reference to the accompanying drawings, in which:

FIG. 1A is a graph illustration of an electrostatic field of cross section of a blood vessel having a damaged wall section;

FIG. 1B is a graph illustration the cross section of the blood vessel of FIG. 1A with a stent having a coating with a negative electric charge;

FIG. 2A is a graph illustration of an electrostatic field of cross section of another blood vessel having a damaged wall section;

FIG. 2B is a graph illustration the cross section of the blood vessel of FIG. 2A with a stent having a coating with a negative electric charge;

FIG. 3 is a schematic illustration of a section of a stent having a coating with a negative electric charge;

FIG. 4A is a front perspective view of a heat sink for mounting thereon a stent;

FIG. 4B is a side view of the heat sink of FIG. 4A; and

FIG. 4C is a top view of the heat sink of FIG. 4A.

### DETAILED DESCRIPTION OF EMBODIMENTS

An objective of the present invention is to provide an implant which restores the body conditions of healthy tissues, in particular, an implant which is configured to restore an electrostatic field formed by healthy tissues of an organ in which the implant is disposed.

It is appreciated that one of the main causes of thrombosis and neo-intimal cell hyperplasia resulting in restenosis is that the electrostatic potential of the intima surface changes. Healthy intima surface in blood vessels is part of a homeostasis range of negative electrostatic filed. The potential of blood cells is also negative, owing to which the physical state of blood is stable and it freely flows in blood vessels. Any damage of blood vessel walls changes the electrostatic charge of the damaged section from negative to positive. As a result, negatively charged platelets are attracted to the positively charged damaged section of the blood vessel, which leads to aggregation of platelets red blood cells and smooth muscles cells and subsequent thrombosis.

Furthermore, the local positive charge stimulates restenosis over the damaged section, which eventually narrows the blood vessel lumen because the growth rate of the intima is proportional to the potential difference into damaged and healthy areas.

Stenting of the narrowed (Atherosclerosis) segment of the blood vessel traumatizes the intima even more and accelerates the processes of thrombosis and restenosis.

An additional source of blood clots may be the design features of the stents. The points of intersection or connection of structural elements are prone to aggregation of blood particles due to their stagnation and capillary effects leading to formation of blood clots.

Accordingly, the implant of the presently disclosed subject matter is aimed to compensate for the positive electrostatic charge caused by the damaged tissue and to restore the electrical homeostasis of the organ in which the implant it disposed. That is to say, the implant is configured to compensate for the influence of the positive electrostatic charge of the damaged tissue which causes a change in the overall electrostatic field in the damaged site inside the cardiovascular organ. The change in the electrostatic field can result in a positive electrostatic field, or even in a negative electrostatic field which is significantly different than the electrostatic field in corresponding healthy tissue, to the extent that thrombosis and restenosis is stimulated.

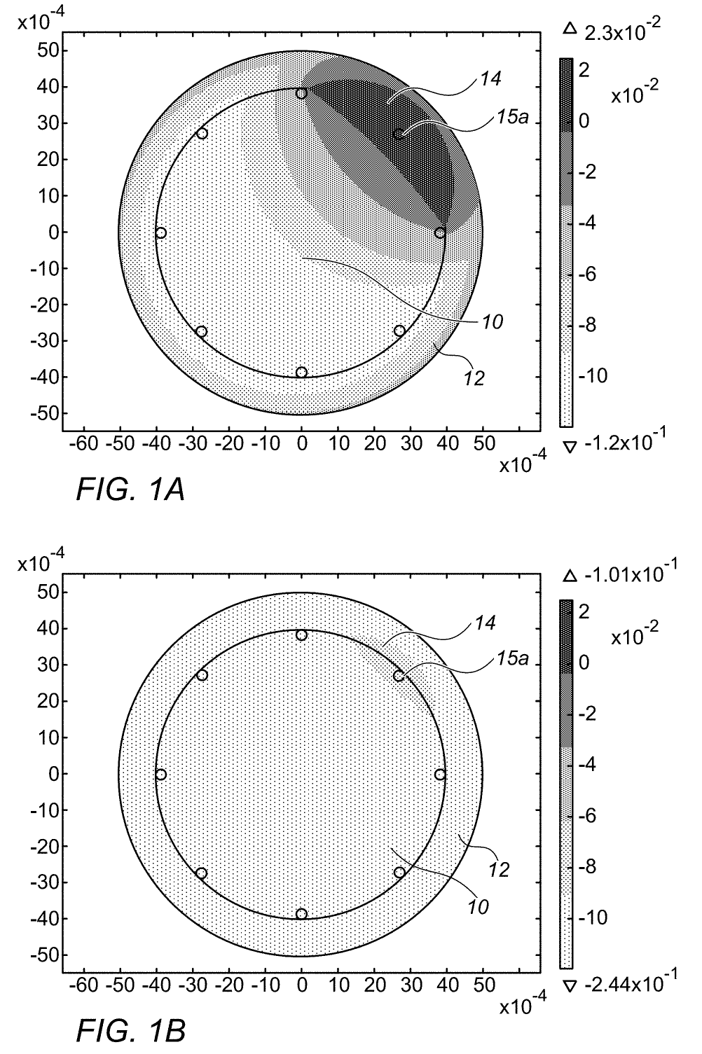

FIGS. 1A and 1B illustrate a cross section of a blood vessel 10 having a wall 12, a portion 14 of which is damaged, for example by mechanical forces exerted by a vascular stent 15a mounted therein. As shown, the inner portion of the blood vessel 10 includes a negative electrostatic field formed by the healthy tissue of the blood vessel wall 12, while the area of the damaged portion 14 includes a positive electrostatic field, or an electrostatic field which is less negative than other areas of the blood vessel.

As shown in FIG. 1B, when a vascular stent 15b having an electric negative charge is inserted into the blood vessel 10, the negative charge forms an electrostatic field which overcomes the positive charge caused by the damaged portion 14 of the wall 12, such that the sum electrostatic field inside the blood vessel is entirely negative and corresponds to an electrostatic field of a healthy blood vessel. The electrostatic field inside the damaged area of the blood vessel can be such that the differences between the electrostatic field in damaged areas and the electrostatic field in healthy areas in close proximity to the damaged areas are regulated, precluding thereby undesirable electric forces acting on platelets and red blood cells and subsequent thrombosis and restenosis.

According to the illustrated example the stent 15b is coated with a coating having an electric charge of

$$- 1 \cdot {10}^{- 8} \frac{c}{{m}^{2}} .$$

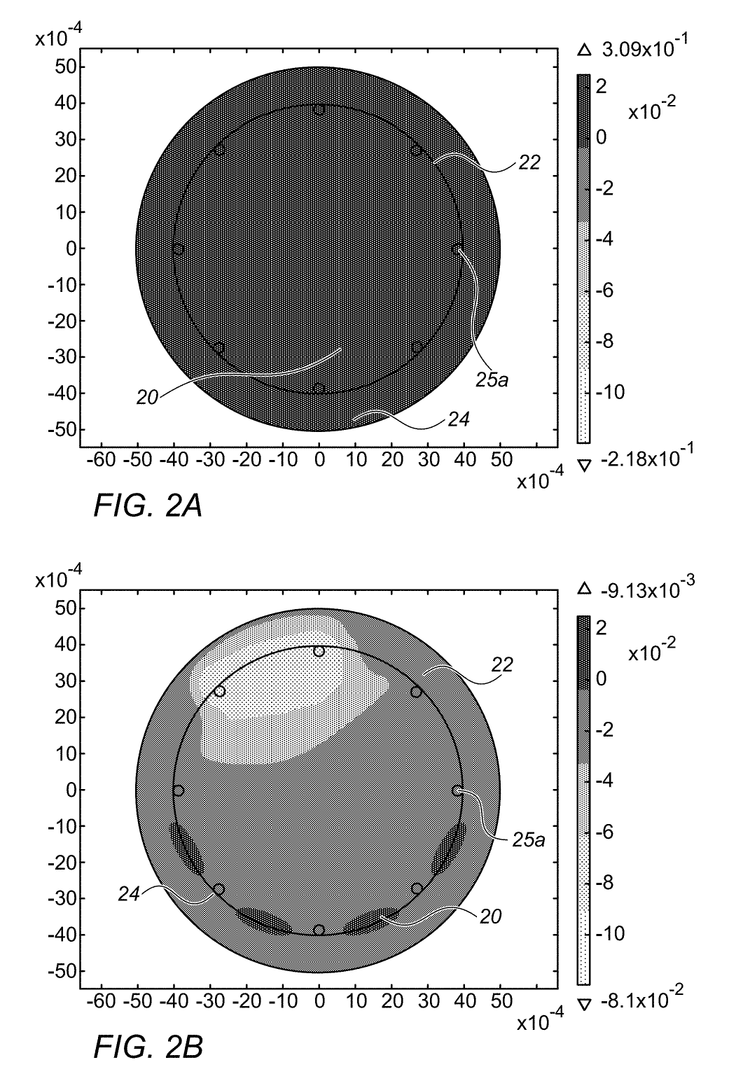

FIGS. 2A and 2B illustrate a cross section of another blood vessel 20 having a wall 22. According to this example the majority of circumference of the wall is damaged and thus radiates a positive electrostatic field into the blood vessel. Due to the large surface area of the damaged wall portion 24, the electrostatic field inside the blood vessel is mostly positive, which hinders the ability of the body to perform a regulated and balanced healing process of the blood vessel wall.

As shown in FIG. 2B, a stent 25b having a negative charge is inserted inside the blood vessel 20, such that the sum electrostatic field inside the blood vessel is entirely negative and corresponds to an electrostatic field of a healthy blood vessel, as described hereinabove.

According to the illustrated example the stent 25b is coated with a coating having an electric charge of

$$- 3 \cdot {10}^{- 8} \frac{c}{{m}^{2}} .$$

It is appreciated that the magnitude of the negative charge can be configured in accordance with the positive charge of the damaged tissue and in accordance with the negative electrostatic field in a corresponding healthy organ. In other words, the amount of negative charge inserted into the coating of the stent, can be in accordance with the size of the damaged area in the blood vessel or an estimation thereof. Thus, in case of a patient having a high risk for damage in the wall of the blood vessel, such as when the flexibility of the wall of the blood vessel is accepted to be relatively low, a stent having a higher negative charge can be used, so as to form a corrective electrostatic field inside the blood vessel.

It is appreciated that as known in the art, the range of electric field in a healthy blood vessel is −50 mv to −400 mv, thus the stent inserted into a blood vessel should have a negative charge configured to restore similar electric field. According to an example, it is estimated that the negative charge in the wall of a healthy blood vessel is about

$$- 1 \cdot {10}^{- 8} \frac{c}{{m}^{2}} ,$$

while the positive charge in the wall of a damaged blood vessel

$$+ 1 \cdot {10}^{- 8} \frac{c}{{m}^{2}} .$$

Thus, in order to correct the electrostatic field inside the damaged blood vessel, the stent can include a negative charge of

$$- 2 \cdot {10}^{- 8} \frac{c}{{m}^{2}} .$$

It would be appreciated by those skilled in the art that the charge in the coating can be selected depending on the type of vessel and the degree of its damage. I.e. the greater the degree of damage, the greater the charge which is required, so as to mitigate the effect of the positive charge of the damaged tissue. That being said, the charge in the coating is in a specific range, such that the hemostasis is maintained and the blood flow is not interrupted. In accordance with an example the range of the charges to be selected can be between

$$- 4 \cdot {10}^{- 8} \frac{c}{{m}^{2}} and - 4 \cdot {10}^{- 7} \frac{c}{{m}^{2}} .$$

In order to estimate the damage of the blood vessel wall, and thus the required corrective electrostatic field, number of parameters can be considered including: the damage caused by the balloon angioplasty, the friction between elements of the stent and the wall of the vessel, background disease, such as diabetes, expected inflammation and stenosis level.

It is appreciated that in case of an implant and other cardiovascular organs, such as an artificial heart valve, the negative charge in the coating of the implant can be configured in accordance with the desired electrostatic filed in the organ in which the implant is to be inserted, such as the heart.

It is further appreciated that in the case of a vascular stent, the stent can be of high biological compatibility including hemocompatibility and temporally stable anti-coagulating and anti-clotting properties implantable into coronary or other arteries to restore patency thereof. The vascular stent can include a cylindrical celled frame made of at least one wire member is coated with a coating having an electret structure. The aforesaid coating carries a negative surface charge and forms thereby a corrective negative electrostatic field.

The electret coating can have an integral surface blanketing an entire surface of the stent. In other words, there is a negative potential on entire surface of the stent. Hence, external stent surfaces directed to the vessel wall and internal stent surfaces directed to an internal space thereof carry a negative electric charge. This results in a negative electrostatic field radiating across the entire area of the vessel along the stent.

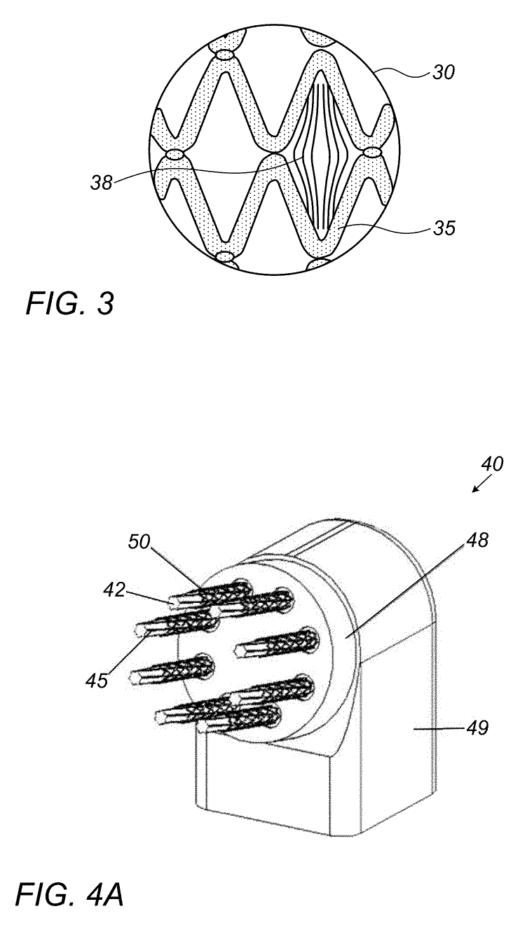

As shown in FIG. 3, the vascular stent 30 can include a mesh body structure having a plurality of wires 35 arranged to form cylindrical body. Each of the wires 35 is coated with a coating having a negative electric charge forming thereby a static negative electric field in the inner volume of the cylindrical body. Since the electrostatic field depends on the distance from the electric charge, as indicated by pattern of the electric field lines 38, the wire 35 can be arranged such that the entire area between each wire has a negative electric filed. I.e. the electric field inside the entire volume of the blood vessel is negative, such that the positive field radiated by the damaged vessel wall is corrected by the negative field. This way, the entire area of the stent is protected from accumulation of thrombosis and the electrostatic field restores the electrostatic field of a corresponding healthy tissue.

According to an example the distance between two neighboring wires is less than 15 times the diameter of the wire. This way, the amount of negative electric charge carried by each wire is sufficient to form a negative electric field which covers the entire area between the wires.

According to an example the electret structure is formed by means of coating the stent with dielectric material provided with high electrical resistance such as tantalum pentoxide (Ta₂O₅) etc.. Negative charge carried by the vascular stent coated with electret films prevents blood plates such as thrombocytes or leucocytes from aggregation at a stent mounted within a blood vessel. It is known that the blood cells to be aggregated at the stent are negatively charged. Electronegativity of the stent surface results in prevention of blood cell aggregation both at the stent per se and the vascular wall-adjacent area due to repelling Coulomb forces. Thus, the negative surface charge generates a negative electrostatic field shielding areas potentially prone to blood cell aggregation and decreases risk of restenosis. Life quality and longevity of the patients stented with temporally stable anti-coagulating and anti-clotting properties are improved in comparison with the unstented patient.

Moreover, as described above, the negative electrostatic field corrects the positive filed radiated by the damaged vessel wall, and restores the negative electrostatic field of a corresponding healthy vessel wall.

The stents provided with tantalum coating are characterized by higher reagent resistance in comparison with the gold coated stents. Tantalum pentoxide is dielectric and characterized by high mechanical properties. Tantalum pentoxide is chemically inert and biologically compatible. Thus, the stent provided with tantalum pentoxide coating is safely implantable into blood vessel and may reside in situ for a long time without expected side effects such as toxicity from stent degradation products and repeat thrombosis at the stent.

It is appreciated that the negative electric charge in the coating of the stent decays overtime, thus, according to an example the process of forming the coating can include configuring the coating such that the negative charge therein is relatively stable. On the other hand, the positive electric charge carried by the damage vessel wall varies over time, such that the overall positive electric field is diminished together with the healing process of the vessel wall. Thus, in accordance with an example, the decay rate of the electrostatic charge in the coating can correspond to the healing process of the vessel wall. This way, the rate at which the coating losses the negative electric charge correspond to the rate at which the blood vessel wall losses the positive electric charge. As a result the electric field inside the blood vessel remains substantially stable and the negatively thereof is always maintained at a range which mimics the electric field of a healthy blood vessel.

Accordingly, the size of the charge can be selected in accordance with the installation site, the required duration of the electrostatic field, the rate of decrease in charge over time, which is characteristic of the conditions at a given location of the stent installation in a particular vessel.

For example, the required electrostatic field in the installation site inside the blood vessel a year after the installation of the stent can be such that the required charge in the coating is about

$$- 1 \cdot {10}^{- 8} \frac{c}{{m}^{2}} .$$

Thus, if the rate of decrease of charge due is such that over the 12 months period only ⅛ of the charge will be maintained in the coating, the initial charge of the stent should be about

$$- 8 \cdot {10}^{- 8} \frac{c}{{m}^{2}} .$$

The following is a description of an exemplary method for forming the tantalum oxide coating having electret structure.

The body structure of the implant is coated with vacuum-plasma sputtering, i.e. the body structure is placed inside a sputtering system which ejects particles of tantalum pentoxide onto the implant together with negatively charged particles, such as electrons or ions. Since the surface of the tantalum pentoxide includes defects in the crystalline structure thereof, the negatively charged particles sprayed on the implant are placed inside the defects. The implant is coated with a number of layers, such that when the negatively charged particles are stored in the defects of the first layer, the second layer of tantalum pentoxide covers the negatively charged particles stored in the defects of the first layer and holds negative charge inside the coating. This way, the negative charge of the coating is maintained in a relatively stable state. It is appreciated that controlling the stability of the negative charge inside the coating is carried out in accordance with the desired lifetime of the negatively charged coating, i.e. the required negative electric filed to serve as a corrective electric filed to counter the positive electric charge of the damaged tissue.

According to an example, the sputtering is carried out such that the temperature of the implant does not exceed a predefined threshold, i.e. a threshold at which the negatively charged particles can no longer be stably stored inside the defects. According to an example the threshold is 100°-120° C., and is maintained by controlling the ejection speed of the tantalum pentoxide and the electrons. In order to better control the direction of the ejected tantalum pentoxide and the electrons a metal having an electric potential can be disposed behind the implant urging the ejected tantalum pentoxide and the electrons in the desired direction. This way, ejection at a low speed can be utilized without compromising on the consistency and accuracy of the coating. It is appreciated that the consistency and accuracy of the coating is such which provides a uniformly spread negative charge and as a result the desired homogenous electric field.

According to a further example, a heat sink can be used for holding the implant thereon during the spurting process, evacuating thereby heat from the implant.

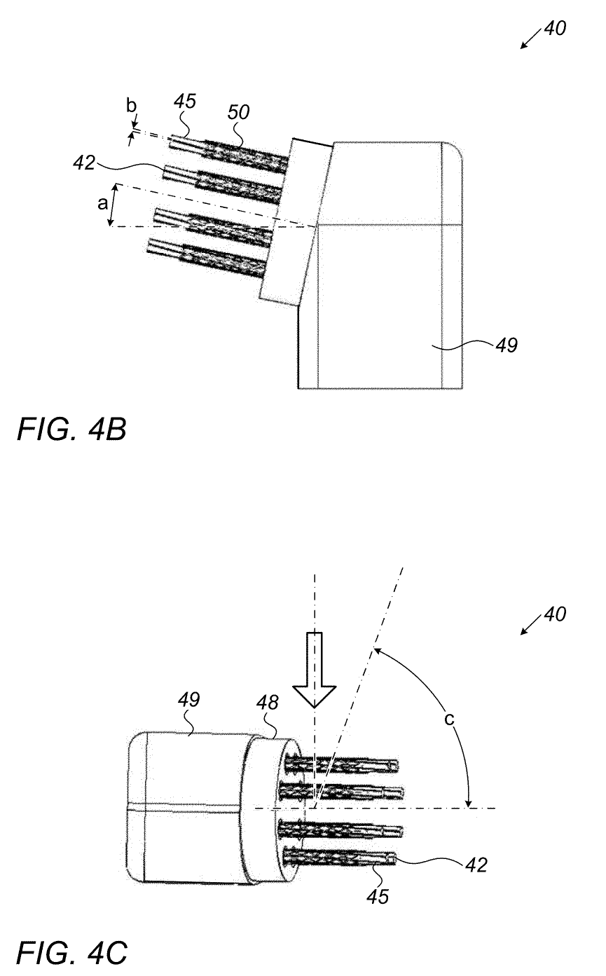

As shown in FIG. 4A to 4C, the heat sink 40 for coating stents can include one or more rotating rods 42 configured for mounting a stent 50 thereon. The rods 42 can have a diameter which is slightly smaller than the inner dimeter of the stent 50. This way, the inner circumference of the wall of the stent does not fully engage the surface of the rod 42 allowing thereby the sputtered particles to reach the inner surface of the stent. According to an example the diameter of the rods 42 is 15-25% smaller than the inner diameter of the stent 50.

Prior to mounting of the stent on the rod 42, the stent can be expended, facilitating thereby coating of all areas of the wires 35 with the plasma coating.

The expansion can be as high as possible so as to improve the access of plasma to the stent. It is however noted that the total plastic deformation of the stent elements after the coating thereof should be as small as possible to ensure a reliable stent performance That is to say, the mounting process of the stent in the desired site is involved expansion thereof. Although the stent is provided with a certain degree of elasticity so as to withstand this expansion, the expansion might cause the stent to undergo an irreversible plastic deformation. Thus, the permissible expansion degree of the stent prior to coating thereof is determined in accordance with the expansion expected to occur during the installation of the stent inside the blood vessel, such that the plastic deformation of the stent elements does not compromise the performance of the stent.

According to an example the expansion rate is approximately 50 to 70% of the nominal diameter of the fully opened stent.

In addition, the coating of tantalum pentoxide can be configured to withstand the deformation of the stent elements without compromising the durability thereof.

The rods 42 can be disposed at an angle with respect to the horizontal plane, such that the stent tends to gravitate downwardly. This way, at the first instance the stent is disposed such that a first half of the inner circumference is disposed on top and engages the rod, while the second half of the inner circumference is disengaged from the rod and is free to be coated. As the rod 42 rotates the stent rotates therewith and the first half of the inner circumference is rotated downwardly, and the second half of the inner circumference is rotated upwardly. At this position the second half of the inner circumference engages the rod while the first half of the inner circumference is disengaged from the rod 42 and is free to be coated.

In addition, as shown in FIG. 2, the rods 42 can include a plurality of ribs 45 extending at an angle b which can be 1-20° to the longitudinal axis of the rods, such that the ribs 45 engages the stent at various locations while exposing other locations thereof to be reached by the coating particles.

Furthermore, the rod 42 can be disposed at an angle a with respect to the horizontal plane, which can be 10-25°.

The rods 42 are anchored perpendicular to the massive disc 48, which can be fixed on the base 49 with the possibility of rotation about its axis perpendicular to the plane of the disc 48. The axis of rotation of the disc and, accordingly, the axis of the rods are is at the angle a.

The base 49 can include a drive for the rotation of the disc 48 at a speed which allows evenly coating the stents for example, 5-60 rpm.

The base 49 can further include a device for cooling the heat sink 40. Cooling can be a liquid, a cooling system based on the Peltier effect or any other cooling system. The disc 48 and rods 42 can be made of a material with a high thermal conductivity.

The heat sink 40 can be disposed with respect to the stream of the particles, with and angle of 70-90° such that the plasma evenly reaches the entire longitudinal dimension of the stent.

Those skilled in the art to which the presently disclosed subject matter pertains will readily appreciate that numerous changes, variations, and modifications can be made without departing from the scope of the invention, mutatis mutandis.

1. An implant for the cardiovascular system, insertable into an organ, the implant comprising:
a body structure configured to be disposed inside an organ;
and an electret coating disposed on said body structure;
wherein said electret coating includes a negative charge such that a negative electrostatic field is formed in proximity of said body structure, said charge is such that said negative electrostatic field corresponds to a positive electrostatic field formed by a damaged tissue of said organ.

2. The implant of claim 1 wherein said negative electrostatic field is configure to restore body conditions of a healthy tissue corresponding to the damaged tissue.

3. The implant of claim 2 wherein said body conditions includes an electrostatic field formed by a healthy tissue of an organ in which the implant is disposed.

4. The implant of claim 1 wherein said negative electrostatic field is configured to compensate for the positive electrostatic charge caused by the damaged tissue and which causes a change in the overall electrostatic field in the damaged site inside the organ.

5. The implant of claim 1 wherein said negative charge is configured to form a sum electrostatic field inside a segment of the organ in which the implant is mounted such that difference between said sum electrostatic field and an electrostatic field in healthy areas in close proximity to said damaged tissue precludes undesirable electric forces acting on platelets and red blood cells.

6. The implant of claim 5 wherein said negative charge is configured in accordance with the size of said damaged area.

7. The implant of claim 6 wherein said negative charge is configured in accordance with the risk level for damaged tissue determined in accordance with the physical conditions of the patient in which the implant is mounted.

8. The implant of claim 1 wherein said negative charge is configured to restore an electrostatic field in the range of −50 mv to −400 mv.

9. The implant of claim 1 wherein said negative charge is selected depending on the type of organ and a degree of said damaged tissue.

10. The implant of claim 1 wherein said negative charge is in a range of
$$- 4 \cdot {10}^{- 8} \frac{c}{{m}^{2}} to - 4 \cdot {10}^{- 7} \frac{c}{{m}^{2}}$$
and is selected such that the hemostasis in the organ is maintained and blood flow therein is not interrupted.

11. The implant of claim 1 wherein said body structure includes a plurality of wires arranged to form a cylindrical body wherein each of said wires is coated with a coating having a negative electrostatic charge forming thereby a negative electrostatic field in the inner volume of said cylindrical body, wherein said wires are arranged such that an entire area between each one of said wires has a negative electrostatic filed.

12. The implant of claim 11 wherein distance between two neighboring wires of said plurality of wires is less than 15 times the diameter of the wire and said negative electric charge carried by each of said wire is sufficient to form a negative electrostatic field which covers the entire area between said wires.

13. The implant of claim 1 wherein said electret coating is formed by means of coating the stent with dielectric material.

14. The implant of claim 1 wherein a decay rate of said negative charge in said coating corresponds to the healing process of the damaged tissue such that the rate at which the coating losses said negative electric charge corresponds to the rate at which the damaged tissue losses the positive electric charge.

15. A method for forming an implant for the cardiovascular system, insertable into an organ, the method comprising:
providing a body structure configured to be disposed inside an organ;
coating said body structure with an electret coating;
wherein said electret coating includes a negative charge such that a negative electrostatic field is formed in proximity of said body structure, said charge is such that said negative electrostatic field corresponds to a positive electrostatic field formed by a damaged tissue of said organ.

16. The method of claim 15 wherein said step of coating is carried out with vacuum-plasma sputtering.

17. The method of claim 16 wherein said vacuum-plasma sputtering includes placing said body structure inside a sputtering system which ejects particles of tantalum pentoxide onto the implant together with negatively charged particles.

18. The method of claim 17 wherein said vacuum-plasma sputtering further includes forming a layer of tantalum pentoxide having defects in a crystalline structure of said layer and targeting said negatively charged particles into said defects.

19. The method of claim 18 wherein said vacuum-plasma sputtering further includes forming at least a first layer and a second layer of tantalum pentoxide, such that negatively charged particles disposed in said defects of said first layer, are covered by said second layer holding said negative charge inside said coating.

20. The method of claim 15 wherein level of stability of said negative charge inside said coating is determined in accordance with a required lifetime of said negative electrostatic filed such that said electrostatic filed compensates for the action of the positive electric charge of the said damaged tissue.

21. The method of claim 17 wherein said vacuum-plasma sputtering is carried out such that temperature of said implant is maintained below a predefined threshold.

22. The method of claim 21 wherein said threshold is defined in accordance with the temperature required to maintain said negatively charged particles negatively charged particles inside said defects.

23. The method of claim 15 wherein said body structure is expandable and wherein said method further comprising expanding said body structure prior to said coating step facilitating thereby coating of all areas of said body structure with the plasma coating.

24. The method of claim 23 wherein said expansion is at a rate determined in accordance with an expansion expected to occur during installation of said implant inside the organ, such that plastic deformation of said body structure does not compromise performance of the implant.

25. The method of claim 15 further comprising placing said body structure on a heat sink prior to said step of coating, wherein said heat sink is configured to evacuate excess heat from said body structure such that said negative charge is maintained in said coating.

26. The method of claim 25 wherein said implant is a stent and said heat sink includes one or more rotating rods configured for mounting thereon said stent.

27. The method of claim 26 wherein diameter of said rod is smaller than the inner dimeter of said stent such that said inner circumference of the wall of the stent does not fully engage the surface of the rod allowing thereby sputtered particles to reach inner surfaces of said stent.

28. The method of claim 26 wherein said rotating rods are disposed at an angle with respect to a horizontal plane of said heat sink such that said stent gravitate downwardly.

29. The method of claim 26 wherein said rotating rods include a plurality of ribs extending at an angle with respect to a longitudinal axis of said rods.

30. The method of claim 26 wherein said rotating rods are anchored perpendicular to a disc.

31. The method of claim 26 wherein said disc is coupled to a base including a cooling device for evacuating heat from said heat sink.

32. The method of claim 25 wherein said step of coating is carried out with vacuum-plasma sputtering in which a stream of particles is sprayed over said implant and wherein said heat sink is disposed at an angle with respect to said stream such that said particles evenly reach a longitudinal axis of said implant.
